(19) United States
(12) Patent Application Publication
(10) Pub. No.: US 2025/0201379 A1
(43) Pub. Date: Jun. 19, 2025

(54) AUTOMATED RECOMMENDATION OF ORTHOPEDIC PROSTHESES BASED ON MACHINE LEARNING

(71) Applicant: Howmedica Osteonics Corp., Mahwah, NJ (US)

(72) Inventors: Manuel Jean-Marie Urvoy, Brest (FR); Emile Ducrocq, Brest (FR); Jérôme Ogor, Brest (FR); Théo Giraudon, Brest (FR)

(21) Appl. No.: 18/844,353

(22) PCT Filed: Mar. 8, 2023
(86) PCT No.: PCT/US2023/014806

§ 371 Date: Sep. 5, 2024

Related U.S. Application Data
(60) Provisional application No. 63/269,025, filed on Mar. 8, 2022.

(51) Int. Cl. G16H 20/40; G06N 20/00; G16H 30/20

(52) U.S. Cl. CPC G16H 20/40; G06N 20/00; G16H 30/20

(57) ABSTRACT

A method for automatically recommending an orthopedic prosthesis for implantation in a patient comprises obtaining, by a computing system, medical image data for the patient; applying, by the computing system, a machine learning model to generate a predicted prosthesis shape for the patient based on the medical image data for the patient; identifying, by the computing system, from a plurality of orthopedic prostheses available for implantation in the patient, an orthopedic prosthesis that corresponds to the predicted prosthesis shape; and recommending, by the computing system, the identified orthopedic prosthesis for implantation in the patient.

This application claims the benefit of U.S. Provisional Patent Application 63/269,025, filed Mar. 8, 2022, the entire content of which is incorporated by reference.

### BACKGROUND

Orthopedic surgeries often involve implanting one or more orthopedic prostheses into a patient. For example, in a total shoulder replacement surgery, a surgeon may attach orthopedic prostheses to a scapula and a humerus of a patient. In an ankle replacement surgery, a surgeon may attach orthopedic prostheses to a tibia and a talus of a patient. When planning an orthopedic surgery, it may be important for the surgeon to select an appropriate orthopedic prosthesis. Selecting an inappropriate orthopedic prosthesis may lead to improperly limited range of motion, an increased probability of failure of the orthopedic prosthesis, complications during surgery, and other adverse health outcomes.

### SUMMARY

This disclosure describes example techniques for automated recommendation of orthopedic prostheses. This disclosure also describes example techniques for automated recommendation of positioning of orthopedic prostheses. As described in this disclosure, a computing system uses a machine learning model to automatically recommend one or more orthopedic prostheses for implantation into a patient. In some examples, the computing system may use a machine learning model to generate a predicted prosthesis shape for a patient. The machine learning model may be trained based on previously planned orthopedic surgeries. After generating the predicted prosthesis shape, the computing system may perform a registration process that identifies, from a plurality of orthopedic prostheses, an orthopedic prosthesis that corresponds to the predicted prosthesis shape. The computing system may then recommend the identified orthopedic prosthesis. In some examples, the computing system may also determine recommended positions of the identified orthopedic prosthesis. The recommended position of the identified orthopedic prosthesis may refer to a position and orientation of the identified orthopedic prosthesis in 3-dimensional (3D) space.

In one example, this disclosure describes a method for automatically recommending an orthopedic prosthesis for implantation in a patient, the method comprising: obtaining, by a computing system, medical image data for the patient, applying, by the computing system, a machine learning model to generate a predicted prosthesis shape for the patient based on the medical image data for the patient; identifying, by the computing system, from data regarding a plurality of orthopedic prostheses available for implantation in the patient, an orthopedic prosthesis that corresponds to the predicted prosthesis shape; and recommending, by the computing system, the identified orthopedic prosthesis for implantation in the patient.

In another example, this disclosure describes a computing system for automatically recommending an orthopedic prosthesis for implantation in a patient, the computing system comprising: a storage system configured to store medical image data for the patient; and processing circuitry configured to: apply a machine learning model to generate a predicted prosthesis shape for the patient based on the medical image data for the patient; identify, from a plurality of orthopedic prostheses available for implantation in the patient, an orthopedic prosthesis that corresponds to the predicted prosthesis shape; and recommend the identified orthopedic prosthesis for implantation in the patient.

In another example, this disclosure describes a computing system comprising means for performing the methods of this disclosure. In another examples, this disclosure describes a non-transitory computer-readable data storage medium comprising instructions that, when executed, cause a computing system to perform the methods of this disclosure.

The details of various examples of the disclosure are set forth in the accompanying drawings and the description below. Various features, objects, and advantages will be apparent from the description, drawings, and claims.

### BRIEF DESCRIPTION OF DRAWINGS

FIG. 1 is a block diagram illustrating an example system that may be used to implement the techniques of this disclosure.

FIG. 2 is a block diagram illustrating example components of a planning system, in accordance with one or more techniques of this disclosure.

FIG. 3 is a conceptual diagram illustrating an example training input image and an expected image of a training dataset, in accordance with one or more techniques of this disclosure.

FIG. 4 is a conceptual diagram illustrating an example production input image and a predicted image, in accordance with one or more techniques of this disclosure.

FIG. 5 is a conceptual diagram illustrating an example operation of a recommendation unit in accordance with one or more techniques of this disclosure.

FIG. 6 is a conceptual diagram illustrating an example convolutional neural network architecture in accordance with one or more techniques of this disclosure.

FIG. 7 is a conceptual diagram illustrating an example point cloud learning model in accordance with one or more techniques of this disclosure.

FIG. 8 is a block diagram illustrating an example architecture of a T-Net model.

FIG. 9 is a conceptual diagram illustrating an example predicted point cloud in accordance with one or more techniques of this disclosure.

FIG. 10 is a block diagram illustrating an example of a machine learning model that includes a point cloud learning model encoder and a decoder, in accordance with one or more techniques of this disclosure.

FIG. 11 is a flowchart illustrating an example operation in accordance with one or more techniques of this disclosure.

FIG. 12 is a conceptual diagram illustrating an example U-Net convolutional neural network in accordance with one or more techniques of this disclosure.

### DETAILED DESCRIPTION

Orthopedic surgeries often involve implanting one or more orthopedic prostheses into a patient. For example, in a total shoulder replacement surgery, a surgeon may attach orthopedic prostheses to a scapula and a humerus of a patient. In an ankle replacement surgery, a surgeon may attach orthopedic prostheses to a tibia and a talus of a patient. In a total knee arthroplasty (TKA) surgery, a surgeon may attach orthopedic prostheses to a femur and tibia of a patient. When planning an orthopedic surgery, it may be important for the surgeon to select an appropriate orthopedic prosthesis. Selecting an inappropriate orthopedic prosthesis may lead to improper range of motion, an increased probability of failure of the orthopedic prosthesis, complications during surgery, and other adverse health outcomes.

Because of the importance of selecting an appropriate orthopedic implant, automated planning systems have been developed to help surgeons select orthopedic prostheses. For instance, in some examples, an automated planning system applies a set of deterministic rules based, e.g., on patient bone geometry, to recommend an orthopedic prosthesis for a patient. However, the accuracy of such automated planning systems may be deficient, and surgeons may lack confidence in the predictions generated by such automated planning systems. Part of the reason for the deficient accuracy and lack of surgeon confidence is that a surgeon may not be certain that the orthopedic prostheses recommended by the automated planning systems are based on cases similar to the patient that the surgeon is planning to treat.

This disclosure describes techniques that may address one or more challenges associated with existing automated planning systems. For instance, in accordance with one or more techniques of this disclosure, a computing system may obtain medical image data for a patient. The computing system may also apply a machine learning model to generate a predicted prosthesis shape for the patient based on the medical image data for the patient. For example, the machine learning model is trained to predict how an orthopedic prosthesis would appear in the medical image data. The computing system may identify, from a plurality of orthopedic prostheses available for implantation in the patient, an orthopedic prosthesis that corresponds to the predicted prosthesis shape. The computing system may recommend the identified orthopedic prosthesis for implantation in the patient. By generating the predicted prosthesis shape using a machine learning model, and then using the predicted prosthesis shape to identify the orthopedic prosthesis from the plurality of orthopedic prostheses, the computing system may avoid the use of deterministic rules. Instead, the computing system may utilize image processing or point cloud processing techniques to generate the predicted prosthesis shape. This may increase the accuracy of the recommendation.

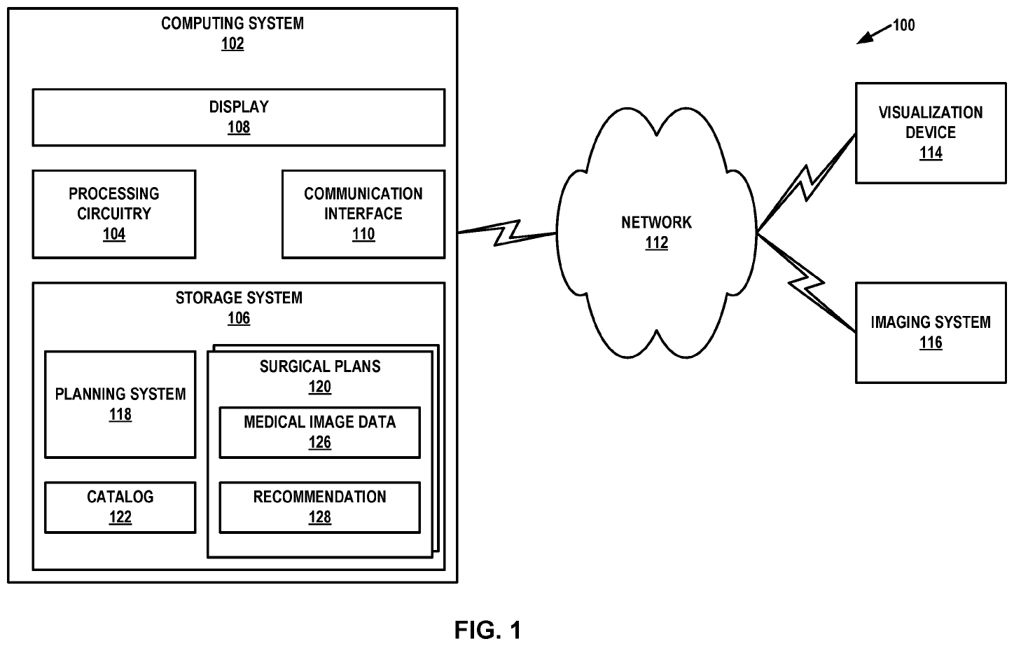

FIG. 1 is a block diagram illustrating an example system 100 that may be used to implement the techniques of this disclosure. FIG. 1 illustrates computing system 102, which is an example of one or more computing devices that are configured to perform one or more example techniques described in this disclosure. Computing system 102 may include various types of computing devices, such as server computers, personal computers, smartphones, laptop computers, and other types of computing devices. In some examples, computing system 102 includes multiple computing devices that communicate with each other. In other examples, computing system 102 includes only a single computing device. Computing system 102 includes processing circuitry 104, storage system 106, a display 108, and a communication interface 110. Display 108 is optional, such as in examples where computing system 102 is a server computer.

Examples of processing circuitry 104 include one or more microprocessors, digital signal processors (DSPs), application specific integrated circuits (ASICs), field programmable gate arrays (FPGAs), discrete logic, software, hardware, firmware or any combinations thereof. In general, processing circuitry 104 may be implemented as fixed-function circuits, programmable circuits, or a combination thereof. Fixed-function circuits refer to circuits that provide particular functionality and are preset on the operations that can be performed. Programmable circuits refer to circuits that can be programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits. In some examples, processing circuitry 104 is dispersed among a plurality of computing devices in computing system 102. In some examples, processing circuitry 104 is contained within a single computing device of computing system 102.

Processing circuitry 104 may include arithmetic logic units (ALUs), elementary function units (EFUs), digital circuits, analog circuits, and/or programmable cores, formed from programmable circuits. In examples where the operations of processing circuitry 104 are performed using software executed by the programmable circuits, storage system 106 may store the object code of the software that processing circuitry 104 receives and executes, or another memory within processing circuitry 104 (not shown) may store such instructions. Examples of the software include software designed for surgical planning, including image segmentation.

Storage system 106 may be formed by any of a variety of memory devices, such as dynamic random access memory (DRAM), including synchronous DRAM (SDRAM), magnetoresistive RAM (MRAM), resistive RAM (RRAM), or other types of memory devices. Examples of display 108 include a liquid crystal display (LCD), a plasma display, an organic light emitting diode (OLED) display, or another type of display device. In some examples, storage system 106 may include multiple separate memory devices, such as multiple disk drives, memory modules, etc., that may be dispersed among multiple computing devices or contained within the same computing device.

Communication interface 110 allows computing system 102 to communicate with other devices via network 112. For example, computing system 102 may output medical images, images of segmentation masks, and other information for display. Communication interface 110 may include hardware circuitry that enables computing system 102 to communicate (e.g., wirelessly or using wires) to other computing systems and devices, such as a visualization device 114 and an imaging system 116. Network 112 may include various types of communication networks including one or more wide-area networks, such as the Internet, local area networks, and so on. In some examples, network 112 may include wired and/or wireless communication links.

Visualization device 114 may utilize various visualization techniques to display image content to a surgeon. In some examples, visualization device 114 is a computer monitor or display screen. In some examples, visualization device 114 may be a mixed reality (MR) visualization device, virtual reality (VR) visualization device, holographic projector, or other device for presenting extended reality (XR) visualizations. For instance, in some examples, visualization device 114 may be a Microsoft HOLOLENS™ headset, available from Microsoft Corporation, of Redmond, Washington, USA, or a similar device, such as, for example, a similar MR visualization device that includes waveguides. The HOLOLENS™ device can be used to present 3D virtual objects via holographic lenses, or waveguides, while permitting a user to view actual objects in a real-world scene, i.e., in a real-world environment, through the holographic lenses. In some examples, there may be multiple visualization devices for multiple users.

Visualization device 114 may utilize visualization tools that are available to utilize patient image data to generate three-dimensional models of bone contours, segmentation masks, or other data to facilitate preoperative planning. These tools may allow surgeons to design and/or select surgical guides and implant components that closely match the patient's anatomy. These tools can improve surgical outcomes by customizing a surgical plan for each patient. An example of such a visualization tool is the BLUEPRINT™ system available from Stryker Corp. The surgeon can use the BLUEPRINT™ system to select, design or modify appropriate implant components, determine how best to position and orient the implant components and how to shape the surface of the bone to receive the components, and design, select or modify surgical guide tool(s) or instruments to carry out the surgical plan. The information generated by the BLUEPRINT™ system may be compiled in a preoperative surgical plan for the patient that is stored in a database at an appropriate location, such as storage system 106, where the preoperative surgical plan can be accessed by the surgeon or other care provider, including before and during the actual surgery.

Imaging system 116 may comprise one or more devices configured to generate medical image data. For example, imaging system 116 may include a device for generating CT images. In some examples, imaging system 116 may include a device for generating MRI images. Furthermore, in some examples, imaging system 116 may include one or more computing devices configured to process data from imaging devices in order to generate medical image data. For example, the medical image data may include a 3D image of one or more bones of a patient. In this example, imaging system 116 may include one or more computing devices configured to generate the 3D image based on CT images or MRI images. In some examples, the medical image data may include a point cloud representing one or more bones of a patient. In this example, imaging system 116 may include one or more computing devices configured to generate the point cloud. Each point in the point cloud may correspond to a set of 3D coordinates of a point on a surface of a bone of the patient. Imaging system 116 may generate the point cloud by identifying the surfaces of the one or more bones in images and sampling points on the identified surfaces. In other examples, computing system 102 may include one or more computing devices configured to generate the medical image data based on data from devices in imaging system 116.

Storage system 106 of computing system 102 may store instructions that, when executed by processing circuitry 104, cause computing system 102 to perform various activities. For instance, in the example of FIG. 1, storage system 106 may store instructions that, when executed by processing circuitry 104, cause computing system 102 to perform activities associated with a planning system 118. In some examples, planning system 118 may be an auto-planning system. For ease of explanation, rather than discussing computing system 102 performing activities when processing circuitry 104 executes instructions, this disclosure may simply refer to planning system 118 or components thereof as performing the activities, or may directly describe computing system 102 as performing the activities.

Additionally, in the example of FIG. 1, storage system 106 stores surgical plans 120 and a catalog 122. Surgical plans 120 may correspond to individual patients. A surgical plan corresponding to a patient may include data associated with a planned or completed orthopedic surgery on the corresponding patient. In the example of FIG. 1, a surgical plan corresponding to a patient may include medical image data 126 for the patient and a recommendation 128 for the patient. In some examples, the surgical plan may include multiple recommendations for the patient. As discussed in greater detail elsewhere in this disclosure, medical image data 126 may include computed tomography (CT) images of bones of the patient or 3D images of bones of the patient based on CT images. In this disclosure, the term “bone” may refer to a whole bone or a bone fragment. In some examples, medical image data 126 may include a point cloud that includes points corresponding to one or more bones of the patient. In some examples, medical image data 126 may include magnetic resonance imaging (MRI) images of one or more bones of the patient or 3D images based on MRI images of the one or more bones of the patient. In some examples, medical image data 126 may include ultrasound images of one or more bones of the patient. Recommendation 128 may be a recommendation of an orthopedic prosthesis to implant in the patient.

Catalog 122 may include data regarding a plurality of orthopedic prostheses. In other words, catalog 122 contains data regarding a plurality of orthopedic prostheses available for implantation in the patient. The orthopedic prostheses may have different predetermined sizes and predetermined shapes. The orthopedic prostheses may include orthopedic prostheses available from one or more medical device suppliers. In some examples, the orthopedic protheses are not patient-specific. In other words, in such examples, shapes and sizes of the orthopedic prostheses are not individually customized to the anatomies of specific patients.

Planning system 118 may be configured to assist a surgeon with planning an orthopedic surgery that involves implantation of an orthopedic prosthesis into a patient. In accordance with one or more techniques of this disclosure, planning system 118 may apply a machine learning model to generate a predicted prosthesis shape for the patient based on medical image data 126 for the patient. The predicted prosthesis shape is a predicted shape of the orthopedic prosthesis. For instance, in some examples, planning system 118 may be configured to generate one or more 2D images or a 3D model of the predicted prosthesis shape. In some examples, such as examples in which medical image data 126 for the patient includes a point cloud, planning system 118 may be configured to generate a point cloud that includes a set of points arranged in the predicted prosthesis shape. Furthermore, planning system 118 may identify, from the plurality of orthopedic prostheses in catalog 122, an orthopedic prosthesis that corresponds to the predicted prosthesis shape. Planning system 118 may recommend the identified orthopedic prosthesis to the surgeon.

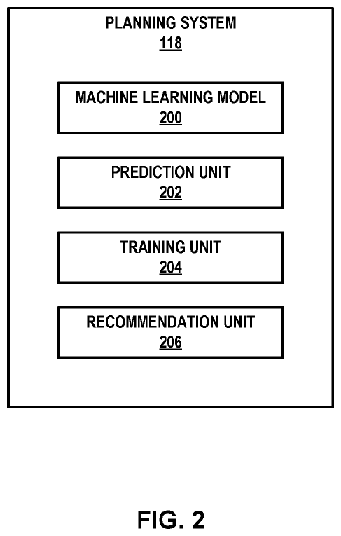

FIG. 2 is a block diagram illustrating example components of planning system 118, in accordance with one or more techniques of this disclosure. In the example of FIG. 2, the components of planning system 118 include a machine learning model 200, a prediction unit 202, a training unit 204, and a recommendation unit 206. In other examples, planning system 118 may be implemented using more, fewer, or different components. For instance, training unit 204 may be omitted in instances where machine learning model 200 has already been trained. In some examples, one or more of the components of planning system 118 are implemented as software modules. Moreover, the components of FIG. 2 are provided as examples and planning system 118 may be implemented in other ways.

Prediction unit 202 may apply machine learning model 200 to generate a predicted prosthesis shape for a patient based on medical image data for the patient. For example, the medical image data for the patient may include a plurality of input images (e.g., CT images or MRI images, etc.). In this example, each of the input images may have a width dimension and a height dimension, and each of the input images may correspond to a different depth-dimension layer in a plurality of depth-dimension layers. In other words, the plurality of input images may be conceptualized as a stack of 2D images, where the positions of individual 2D images in the stack in the depth dimension. In some examples, prediction unit 202 may generate the stack of 2D image (e.g., a 3D image) based on a single 2D image. Furthermore, in this example, prediction unit 202 may provide the input images as input to machine learning model 200. Prediction unit 202 may obtain a plurality of output images generated by machine learning model 200. Each respective output image of the plurality of output images may correspond to a respective depth-dimension layer in the plurality of depth-dimension layers and may show a profile of the predicted prosthesis shape in the respective depth-dimension layer. In some examples, the profile of the predicted prosthesis shape is an outline of an orthopedic prosthesis within an output image. In some examples, the profile of the predicted prosthesis is an opaque or semi-transparent shape within an output image. The output image may also show a recommended position of the predicted prosthesis shape. Thus, predicted prosthesis shapes may appear at different positions within output images. In this disclosure, a recommended position of a predicted prosthesis shape or orthopedic prosthesis may refer to the location and orientation of the predicted prosthesis shape or orthopedic prosthesis in a 3-dimensional space. For instance, the recommended position may have six degrees of freedom (i.e., 3 degrees of freedom for translating the predicted prosthesis shape or orthopedic prosthesis in the 3-dimensional space and 3 degrees of freedom for rotating the predicted prosthesis shape or orthopedic prosthesis in the 3-dimensional space.

In some examples, prediction unit 202 performs one or more preprocessing operations on the input images prior to providing the input images as input to machine learning model 200. In some examples, as part of preprocessing the input images, prediction unit 202 may normalize voxel sizes of the input images, flip one or more of the input images to represent the same laterality, or perform other operations on the input images.

In some examples, the output images include the content of corresponding input image. In other examples, the output images do not include the content of the corresponding input image. Corresponding output and input images may be in the same depth-dimension layer. For instance, in an example where the input images are CT images showing one or more bones of a patient, the output images show the predicted prosthesis shape.

In an example where the medical image data is a point cloud that includes points representing one or more bones of a patient, prediction unit 202 may generate one or more output point clouds that include points representing the one or more bones of the patient and also points representing the predicted prosthesis shape. In other examples where the medical image data is a point cloud that includes points representing one or more bones of the patient, prediction unit 202 may generate one or more output point clouds that include points representing the predicted prosthesis shape but not points that represent the one or more bones. The one or more output point clouds may represent a portion or all of the predicted prosthesis shape at a recommended position of the predicted prosthesis shape. In examples where prediction unit 202 generates more than one output point cloud, two or more of the output point clouds may represent different predicted prosthesis shapes.

In another example, the medical image data for the patient may include a 3D input image, which may be based on CT images, MRI images, or another type of image. The 3D input image may include a 3D matrix of voxels having luminance (e.g., brightness) attributes. In this example, prediction unit 202 may provide the 3D input image as input to machine learning model 200. Prediction unit 202 may obtain one or more 3D output images generated by machine learning model 200. The 3D output image may show the predicted prosthesis shape. The one or more 3D output images may show one or more predicted prosthesis shapes at one or more recommended positions of the predicted prosthesis shape. In examples where prediction unit 202 generates more than one 3D output image, two or more of the 3D output images may represent different predicted prosthesis shapes.

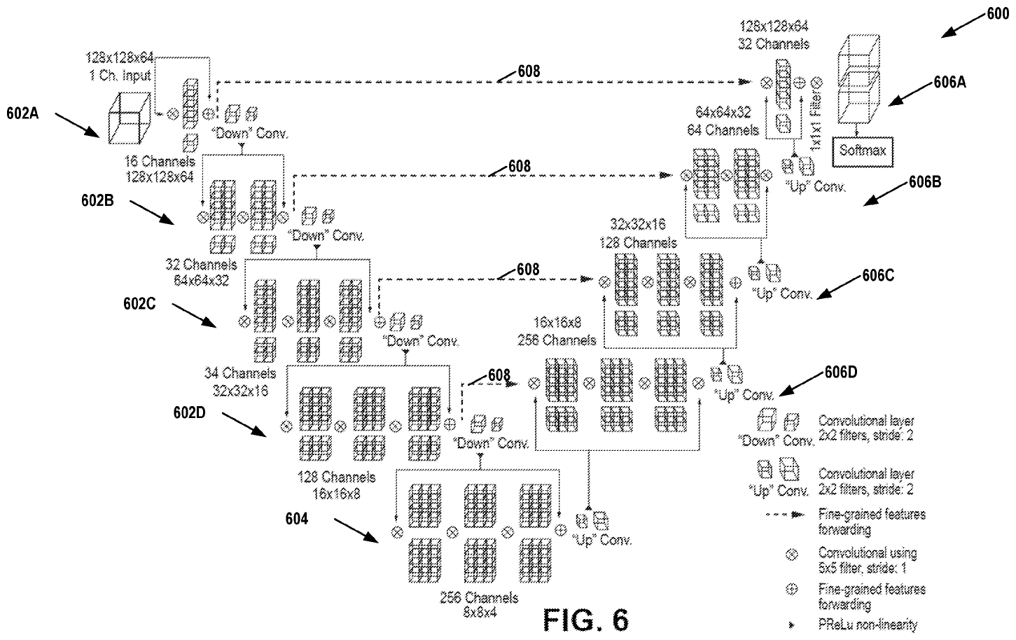

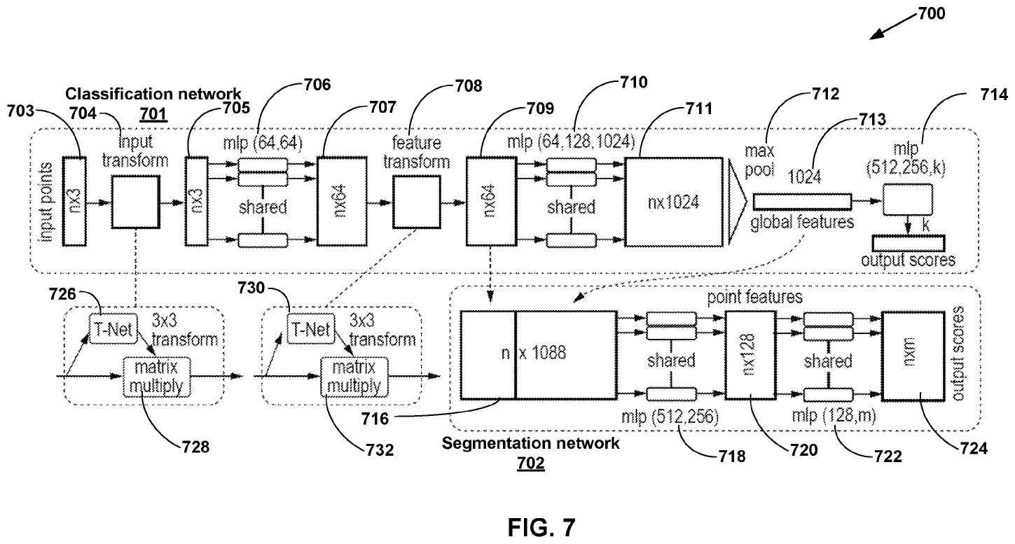

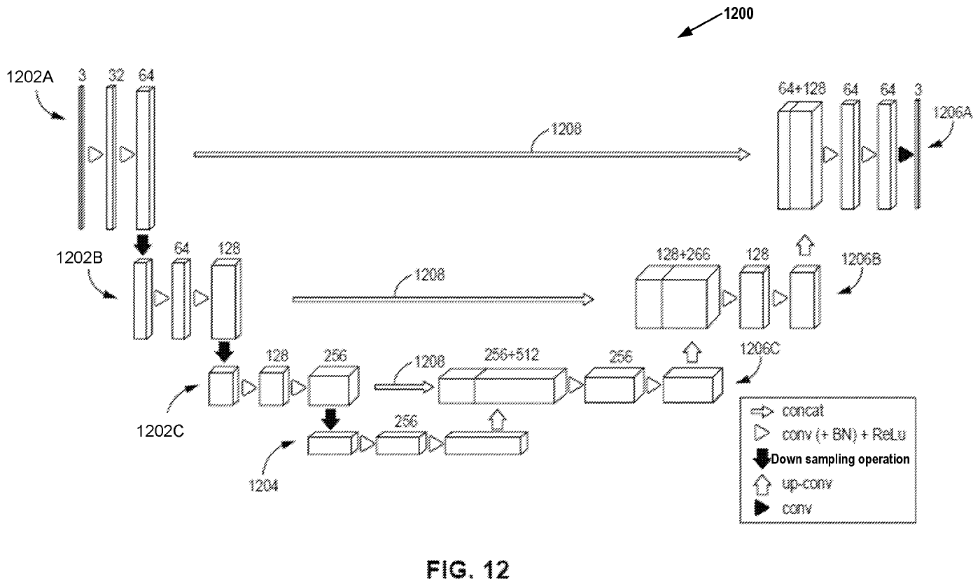

Machine learning model 200 may be implemented in one of a variety of ways. For example, machine learning model 200 may be a convolutional neural network. In some such examples, the convolutional neural network has a U-net or V-net architecture. An example V-net architecture is described with respect to FIG. 6, below. An example U-net architecture is described with respect to FIG. 12, below. The convolutional neural network may generate one or more output images. In examples where the convolutional neural network generates more than one 3D output image, two or more of the 3D output images may represent different predicted prosthesis shapes. In some examples where the medical image data includes a point cloud, machine learning model 200 may be implemented using a point cloud learning model-based architecture. A point cloud learning model-based architecture (e.g., a point cloud learning model) is a neural network-based architecture that receives one or more point clouds as input and generates one or more point clouds as output. In examples where the point cloud learning model generates more than one output point cloud, two or more of the output point clouds may represent different predicted prosthesis shapes. Example point cloud learning models include PointNet, PointTransformer, and so on. An example point cloud learning model-based architecture based on PointNet is described below with respect to FIG. 7.

In some examples, prediction unit 202 may use different machine learning models (e.g., different neural networks) for different types of orthopedic prostheses. For instance, prediction unit 202 may use a first machine learning model for predicting shapes (and, in some examples, recommended positions) of anatomic glenoid implants and a second machine learning model for predicting shapes (and, in some examples, recommended positions) of glenoid reversed implants with patient-specific bone grafts, and so on. Thus, in some examples, prediction unit 202 may apply a first machine learning model to generate a first predicted prosthesis shape for the patient based on the medical image data for the patient, where the first predicted prosthesis shape is a predicted shape of an orthopedic prosthesis in a first type of orthopedic prostheses. Prediction unit 202 may identify, from data regarding a first plurality of orthopedic prostheses available for implantation in the patient, a first orthopedic prosthesis that corresponds to the first predicted prosthesis shape. Recommendation unit 206 may recommend the first identified orthopedic prosthesis for implantation in the patient. Additionally or alternatively, prediction unit 202 may apply a second machine learning model to generate a second predicted prosthesis shape for the patient based on the medical image data for the patient. The second predicted prosthesis shape may be a predicted shape of an orthopedic prosthesis in a second type of orthopedic prostheses. Prediction unit 202 may identify, from data regarding a second plurality of orthopedic prostheses available for implantation in the patient, a second orthopedic prosthesis that corresponds to the predicted prosthesis shape. Each orthopedic prosthesis in the second plurality of orthopedic prostheses is in the second type of orthopedic protheses. Recommendation unit 206 may recommend the second identified orthopedic prosthesis for implantation in the patient. Thus, planning system 118 may provide recommendations for two or more types of orthopedic implants. In some examples, prediction unit 202 may use different machine learning models for different schools of medicine, geographic regions, regulatory regimes, and other factors. Thus, in some examples, prediction unit 202 may use different machine learning models corresponding to different combinations of orthopedic prostheses and factors. In some examples, prediction unit 202 may use a machine learning model that generates multiple predicted prosthesis shapes corresponding to, e.g., different orthopedic prostheses or other factors. For instance, a machine learning model may generate predicted prosthesis shapes corresponding to two or more different schools of medicine.

Training unit 204 may train machine learning model 200. For instance, training unit 204 may generate a plurality of training datasets. Each of the training datasets may correspond to a different historic patient in a plurality of historic patients. The historic patients may include patients for whom surgical plans for implanting orthopedic prostheses have been developed. For instance, surgical plans 120 (FIG. 1) may include surgical plans for the historic patients. In some examples, the surgical plans may be limited to those developed by expert surgeons, e.g., to ensure high quality training data. In some examples, the historic patients may be selected for relevance. The training dataset for a historic patient may include pre-surgical medical image data and expected output data. The pre-implantation medical image data may represent one or more bones of the historic patient prior to implantation of one or more orthopedic prosthesis in the historic patient. In some examples, the pre-surgical medical image data includes one or more Dicom images. In some examples, the expected output data includes the post-surgical medical image data, which may represent shapes of the one or more bones of the historic patient and one or more implanted orthopedic prostheses after implantation of the orthopedic prostheses in the historic patient. In other examples, the expected output data does not include the post-surgical medical image data but includes images of one or more implanted orthopedic prostheses at positions where the orthopedic protheses were implanted in the historic patient.

In some examples, training unit 204 may preprocess images in the training datasets. In some examples, as part of preprocessing the input images, training unit 204 may normalize voxel sizes of the input images, flip one or more of the input images to represent the same laterality, or perform other operations on the input images.

In some examples where planning system 118 is recommending an orthopedic prosthesis for a knee, training unit 204 may obtain different training datasets corresponding to different knee alignment techniques. In the different knee alignment techniques, surgeons may use different techniques for determining how to position knee orthopedic prostheses. For instance, some surgeons may use a mechanical alignment technique to position knee orthopedic prostheses. When a surgeon uses the mechanical alignment technique, the surgeon may position knee orthopedic prostheses perpendicular to mechanical axes of the patient's femur and tibia. Other example techniques may include a kinematic alignment technique and a functional alignment technique. The kinematic alignment technique respects the soft tissue envelope while ignoring the mechanical envelope. The functional alignment technique is a hybrid technique that allows mechanically-sound, soft tissue-friendly alignment targets to be identified and achieved. By training different machine learning models using training sets for different knee alignment techniques, a surgeon may be able to select the knee alignment technique that planning system 118 uses to recommend knee orthopedic prostheses.

In some examples, transfer learning may be used for training the machine learning model 200. For instance, a first machine learning model may be trained for a first type of orthopedic prostheses (e.g., a first type of glenoid implant, a first type of tibial implant, a first type of femoral implant, etc.). Transfer learning may be used for training a second machine learning model for a second type of orthopedic prostheses (e.g., a second type of glenoid implant, a second type of tibial implant, a second type of femoral implant, etc.). Training unit 204 may then train the second machine learning model using training data. Thus, the second machine learning model may be trained in part using transfer learning from the first machine learning model.

The post-surgical medical image data may be generated after an actual surgery to implant the one or more orthopedic prostheses in the historic patient. Alternatively, the post-surgical medical image data may be generated during a planning process for a surgery on the historic patient and may represent how a surgeon expects the one or more bones of the patient and the shapes of the one or more orthopedic prostheses to appear after completion of the surgery. In some examples, the pre-surgical medical image data and post-surgical medical image data includes a plurality of 2D images. In some examples, the pre-surgical medical image data and post-surgical medical image data includes 3D models. In some examples, the pre-surgical medical image data and the post-surgical medical image data includes point clouds. In some examples, expected output data may show a plurality of 2D images or a 3D image of a planned or implanted orthopedic prostheses without showing bone, soft tissue, etc.

Training unit 204 may train machine learning model 200 based on the training datasets. Because training unit 204 generates the training datasets based on how real surgeons actually planned and/or executed surgeries to implant orthopedic prostheses in historic patients, a surgeon who ultimately uses a recommendation generated by planning system 118 may have confidence that the recommendation is based on how other real surgeons selected orthopedic prostheses for real historic patients.

In some examples, as part of training machine learning model 200, training unit 204 may perform a forward pass on the machine learning model 200 using the pre-implantation medical image data of a training dataset as input to machine learning model 200. Training unit 204 may then perform a process that compares the resulting output of machine learning model 200 to the corresponding expected output data. For example, training unit 204 may compare the resulting output of machine learning model 200 to a post-implantation prosthesis binary mask of the training dataset. In this example, the post-implantation prosthesis binary mask may represent a shape (and, in some examples, position) of a planned or implanted orthopedic prosthesis. In some examples, training unit 204 may compare the resulting output of machine learning model 200 to one or more 2D images or a 3D image of the planned or implanted orthopedic prosthesis, which may or may not also show bone or other tissues of a historic patient. Training unit 204 may then perform a backpropagation process based on the comparison to adjust parameters of machine learning model 200 (e.g., weights of neurons of machine learning model 200). Training unit 204 may repeat this process with other training datasets. Training unit 204 may use some of the training datasets for validation of machine learning model 200.

Furthermore, in the example of FIG. 2, recommendation unit 206 may identify, from a catalog 122 (which contains data regarding a plurality of reference orthopedic prostheses available for implantation in the patient), an orthopedic prosthesis that corresponds to the predicted prosthesis shape. For instance, recommendation unit 206 may determine which of the reference orthopedic prostheses in catalog 122 has a shape that most closely matches the predicted prosthesis shape. In some examples, as part of identifying the orthopedic prosthesis, recommendation unit 206 may, for each respective reference orthopedic prosthesis in catalog 122, perform a registration process that aligns the predicted prosthesis shape with the respective reference orthopedic prosthesis. The registration process may generate data representing transforms between a coordinate frame of predicted prosthesis shape and the respective reference orthopedic prosthesis. For purpose of this example, catalog 122 may be considered to be limited to a set of reference orthopedic prostheses. For example, recommendation unit 206 may perform an iterative closest point (ICP) algorithm to align the predicted prosthesis shape with the respective reference orthopedic prosthesis. In this example, as part of performing the ICP algorithm, recommendation unit 206 may generate a source point cloud that represents points in the predicted prosthesis shape and a reference point cloud that represents points in the respective reference orthopedic prosthesis. For each point in the source point cloud, recommendation unit 206 may match the point in the source point cloud to a closest point in the reference point cloud. Recommendation unit 206 may then estimate a combination of rotation and translation using, e.g., a root mean square point-to-point distance metric minimization technique that best aligns each source point to its match found in the previous step. As part of performing this step, recommendation unit 206 may apply weights to points and reject outliers prior to alignment. Recommendation unit 206 may then transform the source points using the obtained transformation. Recommendation unit 206 may iterate through this process multiple times.

Furthermore, recommendation unit 206 may determine a similarity metric for the respective orthopedic prosthesis that indicates a similarity between the aligned predicted prosthesis shape and the respective reference orthopedic prosthesis. In some examples, the similarity metric is a sum of distances between points in the source point cloud and reference point cloud after completion of the ICP algorithm. In some examples, the similarity metric indicates a size of an area of where the predicted prosthesis shape and the respective reference orthopedic prosthesis do not overlap.

In some examples, recommendation unit 206 may align the predicted prosthesis shape with the respective orthopedic prosthesis using a PointNetLK algorithm, e.g., as described in Aoki et al., “PointNetLK: Robust & Efficient Point Cloud Registration using PointNet,” 2019 IEEE/CVF Conference on Computer Vision and Pattern Recognition (CVPR), 2019, pp. 7156-7165, doi: 10.1109/CVPR2019.00733. In some examples, recommendation unit 206 may align the predicted prosthesis shape with the respective orthopedic prosthesis using a process, such as a Nelder-Mead process, that optimizes a Dice Similarity Coefficient (DSC) metric. In an example that uses a Nelder-Mead process, recommendation unit 206 may calculate a DSC that indicates a similarity between a hull defined by a source point cloud (or a 3D image of the predicted prosthesis shape) and a hull defined by a reference point cloud (or a 3D image of a reference prosthesis). Additionally, recommendation unit 206 may apply transformations to the source point cloud to generate a set of 6 additional point clouds. The transformations may rotate and/or translate the source point cloud. Thus, the transformations may change the position of the source point cloud any of 6 degrees of freedom. Hence, a transformation applied to the source point cloud may be expressed as a 6-dimensional vector. In this way, each additional point cloud is associated with a 6-dimensional vector. Recommendation unit 206 may calculate DSC metrics of the source point cloud and the additional point clouds. The DSC metric of a point cloud indicates a similarity between a hull defined by the point cloud and a hull defined by the reference point cloud. Thus, the DSC metric of a point cloud is associated with a location in a 6-dimensional vector space.

Furthermore, recommendation unit 206 may determine an order of seven (7) locations in the 6-dimensional vector space (recall each location in the 6-dimensional vector space is associated with the DSC metric of the source point cloud or a transformed version of the source point cloud). The order of the 7 locations is based on the DSC metrics associated with the 7 locations. The 7 locations form a simplex in the 6-dimensional vector space. Recommendation unit 206 may then determine whether a termination condition is reached. Recommendation unit 206 may determine that the termination condition is reached if a standard deviation of the DSC metrics associated with the 7 locations of the simplex is less than a tolerance threshold. If the optimization process is not to terminate, recommendation unit 206 may calculate a centroid location of all locations of the simplex except for a lowest-ordered location of the simplex.

Furthermore, after calculating the centroid, recommendation unit 206 may compute a reflected location. For instance, recommendation unit 206 may compute the reflected location as xᵣ=x₀+α(x₀−xₙ₊₁) with α>0, where xᵣ is a 6-dimensional vector indicating a reflected location, x₀ is a 6-dimensional vector indicating the centroid location, and xₙ₊₁ is a 6-dimensional vector indicating the lowest-ordered location. Recommendation unit 206 may then apply a transformation to the source point cloud based on the values in the 6-dimensional vector of the reflected location and then determine a DSC metric associated with the location indicated by the 6-dimensional vector of the resulting point cloud. If the DSC metric associated with the location indicated by the 6-dimensional vector of the resulting point cloud is better (e.g., greater) than the DSC metric associated with the second-to-lowest-ordered location of the simplex, but the DSC metric associated with the location indicated by the 6-dimensional vector of the resulting point cloud is not better than the DSC metric associated with the highest-ordered location of the simplex, recommendation unit 206 replaces the lowest-ordered location of the simplex with the reflected location. Recommendation unit 206 may then start a new iteration at the step of ordering the locations of the simplex.

However, if the reflected point is better than the highest-ranked location, recommendation unit 206 may determine an expanded location. For instance, recommendation unit 206 may determine the expanded location xₑ as xₑ=x₀+γ(xᵣ−x₀) with γ>1, where xᵣ is the reflected location and x₀ is the centroid location. Recommendation unit 206 may then determine a DSC metric associated with the expanded location. If the expanded location is better than the reflected location (e.g., the DSC metric associated with the expanded location is greater than the DSC metric of the reflected location), recommendation unit 206 may change the simplex by replacing the lowest-ordered location with the expanded point. If the expanded location is not better than the reflected location, recommendation unit 206 may change the simplex by replacing the lowest-ordered location with the reflected location. In either case, recommendation unit 206 may then start a new iteration at the step of ordering the locations of the simplex.

If the reflected point is better than or equal to the second-to-highest-ranked location, recommendation unit 206 may determine a contracted location. For instance, recommendation unit 206 may determine the contracted location x_{c} as x_{c}=x₀+ρ(xₙ₊₁−x₀) with 0<ρ≤0.5. Recommendation unit 206 may then determine a DSC metric associated with the contracted location. If the contracted location is better than the lowest-ordered location of the simplex, recommendation unit 206 may change the simplex by replacing the lowest-ordered location of the simplex by the contracted location. Recommendation unit 206 may then start a new iteration at the step of ordering the locations of the simplex.

Otherwise, recommendation unit 206 may replace all locations in the simplex except the highest-ordered location with xᵢ=x₁+σ(xᵢ−x₁), where x₁ is the highest-ordered location, xᵢ is a location at order i in the order of locations of the simplex, and σ is a shrink coefficient (e.g., ½ or another value). Recommendation unit 206 may then start a new iteration at the step of ordering the locations of the simplex. Recommendation unit 206 may continue iterating in this manner until the termination condition is reached. The similarity metric may be the DSC metric of the centroid of the simplex when the termination condition is reached.

Recommendation unit 206 may identify the orthopedic prosthesis based on the similarity metrics for the reference orthopedic prostheses. For example, recommendation unit 206 may identify the orthopedic prosthesis as the reference orthopedic prosthesis with the greatest (or lowest) similarity metric. Recommendation unit 206 may recommend the identified orthopedic prosthesis for implantation in the patient. For example, recommendation unit 206 may output an indication of the identified orthopedic prosthesis for display, e.g., on display 108 or visualization device 114 (FIG. 1). In this way, a surgeon may be able to review the identified orthopedic prosthesis.

In some examples, recommendation unit 206 may output for display one or more images (e.g., one or more 2D or 3D images) or models of the identified orthopedic prosthesis at the recommended position. For example, recommendation unit 206 may output one or more images showing the identified orthopedic prosthesis at a recommended location relative to models of one or more bones or other anatomy of the patient. For instance, recommendation unit 206 may output one or more image showing the identified orthopedic prosthesis at a recommended location relative a bones or anatomy of the patient in a 3D CT volume. The identified orthopedic prosthesis, bones, and/or anatomy may be labeled in the images. In some examples, recommendation unit 206 may determine the recommended location of the identified orthopedic prosthesis by performing a process to align a 3D image or point cloud of the identified orthopedic prosthesis with a 3D image or point cloud of the predicted prosthesis shape. For instance, in the example described above the uses a Nelder-Mead process, the transformation that corresponds to the centroid location of the simplex when the termination condition is reached may describe the recommended location of the identified orthopedic prosthesis.

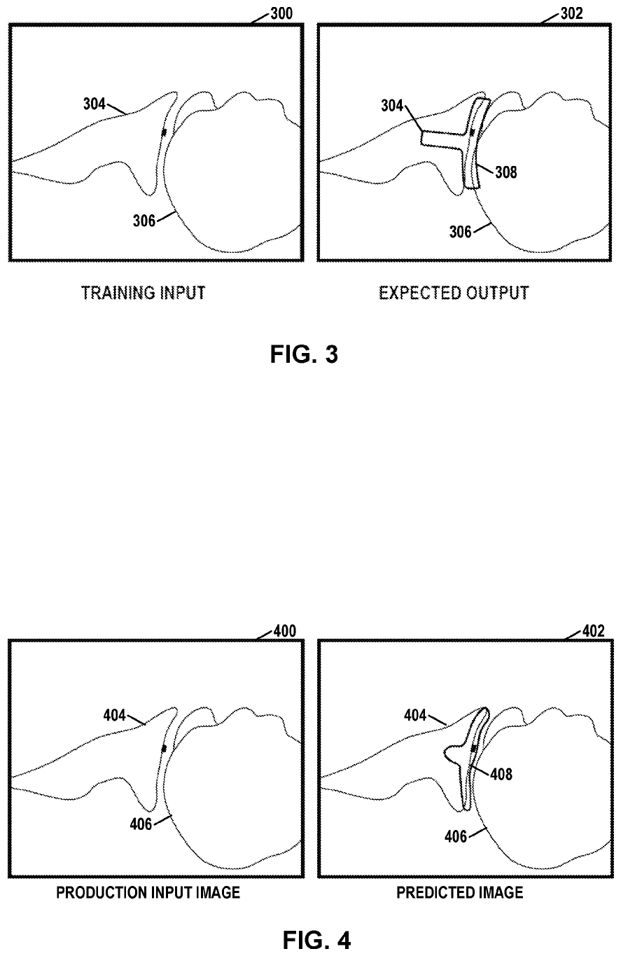

FIG. 3 is a conceptual diagram illustrating an example training input image 300 and an expected image 302 of a training dataset, in accordance with one or more techniques of this disclosure. In the example of FIG. 3, training input image 300 is a pre-implantation CT image of a shoulder joint of a historic patient, with a scapula 304 at the left and a humerus 306 at the right. Although FIG. 3 and the following figures show a scapula, a humerus, and an associated orthopedic prosthesis, the techniques of this disclosure may be applicable to other bones and joints, such as bone of the knee, hip, ankle, elbow, wrist, spine, and so on. Expected image 302 is a post-implantation medical image showing a shape 308 of an orthopedic prosthesis that a surgeon planned to implant or actually implanted into the historic patient.

FIG. 4 is a conceptual diagram illustrating an example production input image 400 and a predicted image 402, in accordance with one or more techniques of this disclosure. Prediction unit 202 may provide production input image 400 to machine learning model 200 when a surgeon is using planning system 118 to obtain a recommendation of an orthopedic prosthesis for implantation in a patient while the surgeon is planning an orthopedic surgery on the patient. This disclosure may use the term “production” to refer to production input image 400 because production input image 400 may be provided as input to machine learning model 200 during a production phase (i.e., after a primary training phase) of machine learning model 200.

Machine learning model 200 may generate predicted image 402 based on production input image 400. In the example of FIG. 4, production input image 400 and predicted image 402 show a scapula 404 and a humerus 406. Furthermore, predicted image 402 shows a predicted prosthesis shape 408. In the example of FIG. 4, predicted prosthesis shape 408 is an outline.

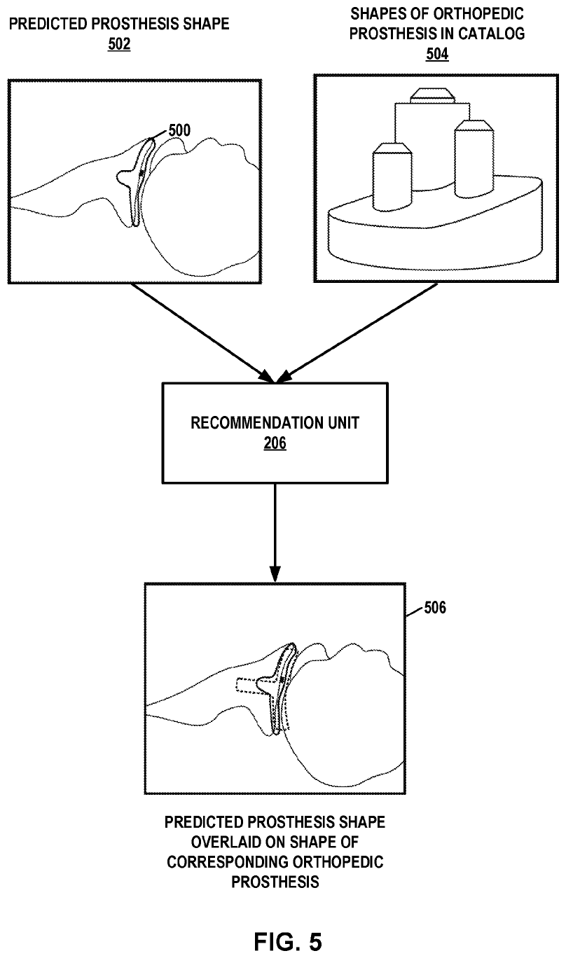

FIG. 5 is a conceptual diagram illustrating an example operation of recommendation unit 206 in accordance with one or more techniques of this disclosure. As shown in the example of FIG. 5, recommendation unit 206 may obtain a predicted prosthesis shape 500, which may be generated by machine learning model 200. In the example of FIG. 5, predicted prosthesis shape is shown in an image 502. Additionally, recommendation unit 206 may obtain shapes 504 of orthopedic prostheses in catalog 122. Recommendation unit 206 may use predicted prosthesis shape 500 and the shapes of orthopedic prostheses to identify an orthopedic prosthesis that corresponds to the predicted prosthesis shape.

FIG. 5 also includes an image 506 showing the predicted prosthesis shape 500 overlaid on the shape of an orthopedic prosthesis identified as corresponding to predicted prosthesis shape 500. In some examples, recommendation unit 206 may generate image 506 for review by a surgeon. Alternatively, image 506 may merely be a concept for purposes of explanation in this disclosure. As shown in image 506, predicted prosthesis shape 500 may not be precisely the same as the shape of the identified orthopedic prosthesis. Although FIGS. 3, 4, and 5 relate to the shoulder, the techniques of this disclosure may be applied to other parts of a patient, such as the patient's knee, hip, ankle, etc. Furthermore, although FIGS. 3, 4, and 5 show 2D images, a 3D predicted prosthesis shape may be shown in a 3D image of the patient's anatomy.

FIG. 6 is a conceptual diagram illustrating an example convolutional neural network (CNN) architecture in accordance with one or more techniques of this disclosure. In the example of FIG. 6, the medical image data may include a 3D image of one or more bones of the patient. Prediction unit 202 (or another system) may generate the 3D image based on a stack of 2D images (e.g., CT images) arranged along a depth dimension. The location of each pixel in the stack of 2D images may be a voxel.

In the example of FIG. 6, a CNN 600 is implemented using a V-net architecture. A left branch of the “V” shape may be referred to as an “encoder.” The encoder extracts high level features in a 3D input image. A right branch of the “V” shape may be referred to as a “decoder.” The decoder reconstructs information at a voxel level. An output of NN 600 includes a 3D output image that includes a 3D predicted prosthesis shape. The 3D output image may have the same size as the 3D input image. In other words, for each voxel of the input image, CNN 600 may output a corresponding voxel.

In the example of FIG. 6, the encoder is composed of a succession of convolution, batch normalization and activation (“ReLU”) layers at each of levels 602A-602D (collectively, “levels 602”). A transition level 604 between the levels 602 of the encoder includes a convolution with a stride of 2 which learns how to reduce the dimension of the feature maps by 2 to focus on most important features. This is denoted as “Down Conv.” in FIG. 6. In the example of FIG. 6, the number of channels (filters) are defined at 16, 32, 64, 128 and 256 for the 5 levels. The convolutions at each level use a 5×5×5 filter with a stride of 1. In other examples, the number of channels, number of levels, filter size, and stride length may be different.

The decoder may be symmetrically built where the “Down” convolutions are replaced by “Up” convolutions, which include doubling the size of the feature maps at each level of levels 606A-606D (collectively, “levels 606”). In some examples, CNN 600 includes bridges 608 that enable connections between some high-level features that are extracted to help the reconstruction at the voxel level in the decoder. The output of the decoder may include a plurality of output images corresponding to a respective depth-dimension layer in the plurality of depth-dimension layers and may show a profile of the predicted prosthesis shape in the respective depth-dimension layer.

Thus, in the example of FIG. 6, the medical image data for the patient includes a plurality of CT images, each of the CT images has a width dimension and a height dimension, and each of the CT images corresponds to a different depth-dimension layer in a plurality of depth-dimension layers. In such examples, as part of applying machine learning model 200 to generate the predicted prosthesis shape for the patient, prediction unit 202 of planning system 118 may provide the CT images as input to machine learning model 200. Prediction unit 202 may obtain a plurality of output images generated by machine learning model 200. Each respective output image of the plurality of output images corresponds to a respective depth-dimension layer in the plurality of depth-dimension layers and shows a profile of the predicted prosthesis shape in the respective depth-dimension layer.

Training unit 204 may train CNN 600 or similar machine learning models. For example, training unit 204 may generate a plurality of training datasets. For each respective training dataset of the plurality of training datasets, the respective training dataset may correspond to a respective historic patient of a plurality of historic patients. Furthermore, the respective training dataset may include a respective set of images showing the bone of the respective historic patient. The respective training dataset includes a set of expected images showing a planned orthopedic prosthesis that was planned for attachment to the bone of the respective historic patient. Training unit 204 may train machine learning model 200 (e.g., CNN 600) based on the training datasets.

FIG. 7 is a conceptual diagram illustrating an example point cloud learning model 700 in accordance with one or more techniques of this disclosure. Point cloud learning model 700 may receive an input point cloud. The input point cloud is a collection of points. The points in the collection of points are not necessarily arranged in any specific order. Thus, the input point cloud may have an unstructured representation.

In the example of FIG. 7, point cloud learning model 700 includes a classification network 701 and a segmentation network 702. Classification network 701 receives an array 703 of n points. The points in array 703 may be the input point cloud of point cloud learning model 700. Each of the points in array 703 has a dimensionality of 3. For instance, in a Cartesian coordinate system, each of the points may have an x coordinate, a y coordinate, and a z coordinate.

Classification network 701 may apply an input transform 704 to the points in array 703 to generate an array 705. Classification network 701 may then use a first shared multi-layer perceptron (MLP) 706 to map each of the n points in array 705 from three dimensions to a larger number of dimensions a (e.g., a=64 in the example of FIG. 7), thereby generating an array 707 of n×a (e.g., n×64 values). For ease of explanation, the following description of FIG. 7 assumes that a is equal to 64 but in other examples other values of a may be used. Classification network 701 may then apply a feature transform 708 to the values in array 707 to generate an array 709 of n×64 values. For each of the n points in array 709, classification network 701 uses a second shared MLP 710 to map the n points from a dimension to b dimensions (e.g., b=1024 in the example of FIG. 7), thereby generating an array 711 of n×b (e.g., n×1024 values). For ease of explanation, the following description of FIG. 7 assumes that b is equal to 1024 but in other examples other values of b may be used. In some examples, classification network 701 applies a max pooling layer 712 to generate a global feature vector 713. In the example of FIG. 7, each of points n in global feature vector 713 has 1024 dimensions.

A fully-connected network 714 may map global feature vector 713 to k output classification scores. The value k is an integer indicating a number of classes. Each of the output classification scores corresponds to a different class. An output classification score corresponding to a class may indicate a level of confidence that the input point cloud as a whole corresponds to the class. Fully-connected network 714 includes a neural network having two or more layers of neurons in which each neuron in a layer is connected to each neuron in a subsequent layer. In the example of FIG. 7, fully-connected network 714 includes an input layer having 512 neurons, a middle layer having 256 neurons, and an output layer having k neurons. In some examples, fully-connected network 714 may be omitted from classification network 701.

Input 716 to segmentation network 702 may be formed by concatenating the n 64-dimensional points of array 709 with global feature vector 713. In other words, for each point of the n points in array 709, the corresponding 64 dimensions of the point are concatenated with the 1024 features in global feature vector 713. Shared MLP 718 lowers the dimensionality of the n points (e.g., to 128 in the example of FIG. 7), thereby generating an array 720 of n×128 values. MLPs 722 further lower the dimensionality of the n points to m, where m is the number of segmentation classes. In this way, segmentation network 702 may generate an array 724 of n×m. In some examples of this disclosure, the m is equal to 2 and the two segmentation classes may correspond to “bone” and “orthopedic prosthesis.” Thus, in such examples, point cloud learning model 700 may be configured to determine which points in the input point cloud are associated with the bone of the patient and which points in the input point cloud are associated with an orthopedic implant. The actual values in array 724 may include a set of m values for each of the n points. Each of the m values for a point corresponds to a different class and may indicate a level of confidence that the point corresponds to the class.

In some examples, input 716 to segmentation network 702 (which may also be referred to as decoder network 702) may be formed by concatenating the n 64-dimensional points of array 709 with global feature vector 713. In other words, for each point of the n points in array 709, the corresponding 64 dimensions of the point are concatenated with the 1024 features in global feature vector 713. In some examples, array 709 is not concatenated with global feature vector 713.

In some examples, decoder network 702 (which is an example of segmentation network 702) may sample N points in a unit square in 2-dimensions. Thus, decoder network 702 may randomly determine N points having x-coordinates in a range of [0,1] and y-coordinates in the range of [0,1]. For each respective point of the N points, decoder network 702 may obtain a respective input vector by concatenating the respective point with global feature vector 713. Thus, in examples where array 709 is not concatenated with global feature vector 713, each of the input vectors may have 1026 features. For each respective input vector, decoder network 702 may apply each of K MLPs 718 (where K is an integer greater than or equal to 1) to the respective input vector. Each of MLPs 718 may correspond to a different patch (e.g., area) of the output point cloud. When decoder network 702 applies the MLP to an input vector, the MLP may generate a 3-dimensional point in the patch (e.g., area) corresponding to the MLP. Thus, each of the MLPs 718 may reduce the number of features from 1026 to 3. The 3 features may correspond to the 3 coordinates of a point of the output point cloud. For instance, for each sampled point n in N, the MLPs 718 may reduce the features from 1026 to 512 to 256 to 128 to 64 to 3. In this example, array 720 and MLPs 722 may be omitted. Thus, decoder network 302 may generate a K×N×3 vector in array 724 containing an output point cloud. In some examples, K=16 and N=512, resulting in second point cloud with 8192 3D points. In other examples, other values of K and N may be used. In some examples, as part of training the MLPs of decoder network 302, decoder network 702 may calculate a chamfer loss of an output point cloud relative to a ground-truth point cloud. Decoder network 702 may use the chamfer loss in a backpropagation process to adjust parameters of the MLPs. In this way, planning system 118 may apply the decoder (e.g., decoder network 302) to generate the premorbid bone model based on the global feature vector.

In some examples, MLPs 318 may include a series of four fully-connected layers of neurons. For each of MLPs 318, decoder network 302 may pass an input vector of 1026 features to an input layer of the MLP. The fully-connected layers may reduce to number of features from 1026 to 512 to 256 to 3.

Input transform 704 and feature transform 708 in classification network 701 may provide transformation invariance. In other words, point cloud learning model 700 may be able to classify points in the input point cloud in the same way, regardless of how the input point cloud is rotated, scaled, or translated. The fact that point cloud learning model 700 provides transform invariance may be advantageous because it may reduce the susceptibility of prediction unit 202 to errors based on positioning/scaling in medical image data 126 (FIG. 1). As shown in the example of FIG. 7, input transform 704 may be implemented using a T-Net model 726 and a matrix multiplication operation 728. T-Net model 726 generates a 3×3 transform matrix based on array 703. Matrix multiplication operation 728 multiplies array 703 by the 3×3 transform matrix. Similarly, feature transform 708 may be implemented using a T-Net model 730 and a matrix multiplication operation 732. T-Net model 730 may generate a 64×64 transform matrix based on array 707. Matrix multiplication operation 728 multiplies array 707 by the 64×64 transform matrix.

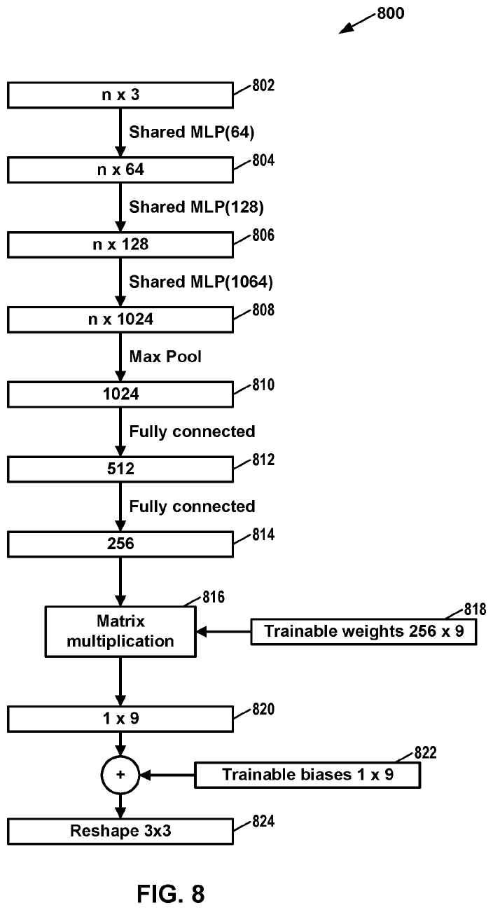

FIG. 8 is a block diagram illustrating an example architecture of a T-Net model 800. T-Net model 800 may implement T-Net model 726 used in the input transform 704. In the example of FIG. 8, T-Net model 800 receives an array 802 as input. Array 802 includes n points. Each of the points has a dimensionality of 3. A first shared MLP maps each of the n points in array 802 from 3 dimensions to 64 dimensions, thereby generating an array 804. A second shared MLP maps each of the n points in array 804 from 64 dimensions to 128 dimensions, thereby generating an array 806. A third shared MLP maps each of the n points in array 806 from 128 dimensions to 1024 dimensions, thereby generating an array 808. T-Net model 800 then applies a max pooling operation to array 808, resulting in an array 810 of 1024 values. A first fully-connected neural network maps array 810 to an array 812 of 512 values. A second fully-connected neural network maps array 812 to an array 814 of 256 values. T-net model 800 applies a matrix multiplication operation 816 to a matrix of trainable weights 818. The matrix of trainable weights 818 has dimensions of 256×9. Thus, multiplying array 814 by the matrix of trainable weights 818 results in an array 820 of size 1×9. T-net model 800 may then add trainable biases 822 to the values in array 820. A reshaping operation 824 may remap the values resulting from adding trainable biases 822 into a 3×3 transform matrix. In other examples, the sizes of the matrixes and arrays may be different.

T-Net model 730 (FIG. 7) may be implemented in a similar way as T-Net model 800 in order to perform feature transform 708. However, in this example, the matrix of trainable weights 818 is 256×4096 and the trainable biases 822 has size 1×4096 bias values instead of 9. Thus, the T-Net model for performing feature transform 708 may generate a transform matrix of size 64×64. In other examples, the sizes of the matrixes and arrays may be different.

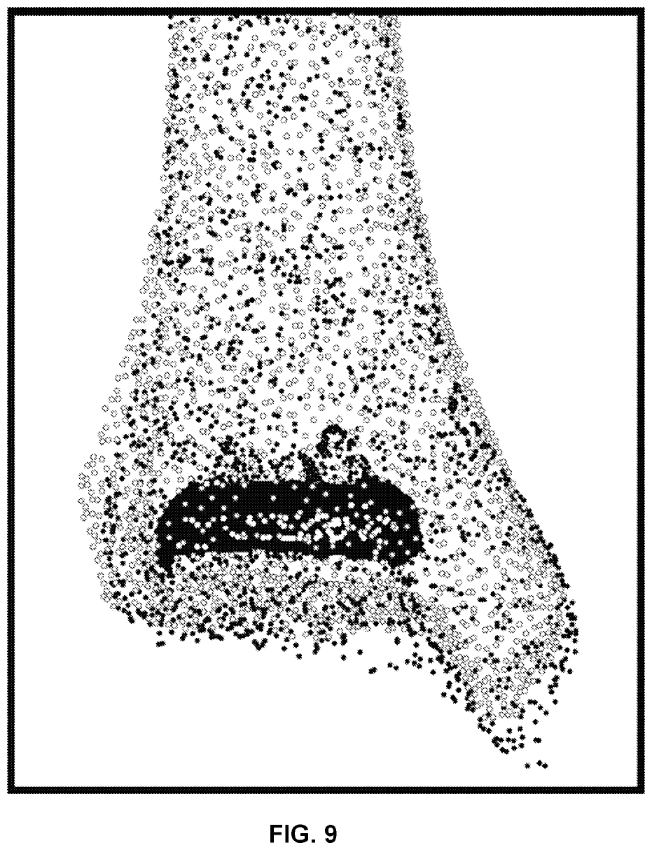

FIG. 9 is a conceptual diagram illustrating an example predicted point cloud 900 generated by point cloud learning model 700, in accordance with one or more techniques of this disclosure. In the example of FIG. 9, hollow dots correspond to points of an input point cloud and filled dots correspond to points of an output point cloud. As can be seen in the example of FIG. 9, the points of the output point cloud include points corresponding to a shape of an orthopedic prosthesis positioned at a recommended location relative to a tibia. Other examples may apply to other bones. Because the output point cloud includes points corresponding to the bone and points corresponding to the orthopedic prosthesis, prediction unit 202 may segment the output point cloud to identify points of the output point cloud that correspond to the bone and points of the output point cloud that correspond to the orthopedic prosthesis. In some examples, prediction unit 202 may use segmentation network 702 to segment the output point cloud. In other examples, point cloud learning model 700 may be configured such that the output point cloud only includes points corresponding to the orthopedic prosthesis and not the bone.

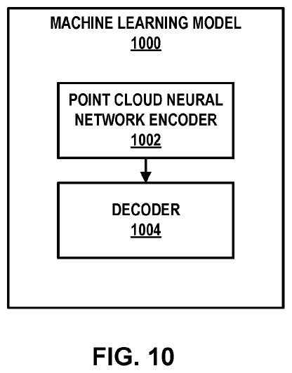

FIG. 10 is a block diagram illustrating an example of machine learning model 1000 that includes a point cloud learning model encoder 1002 and a decoder 1004, in accordance with one or more techniques of this disclosure. Machine learning model 1000 may be an example of machine learning model 200 (FIG. 2). Prediction unit 202 may obtain an input point cloud for machine learning model 1000 based on pre-implantation medical images of a patient for whom a surgeon is planning a surgery. Thus, the input point cloud for machine learning model 1000 does not include points representing an orthopedic prosthesis.

Point cloud neural network encoder 1000 and decoder 1002 may operate as an auto-encoder. Point cloud neural network encoder 1002 operates as the encoder branch of the auto-encoder. Decoder 1004 operates as the decoder branch of the auto-encoder. In this example, point cloud learning model encoder 1002 may correspond to the classification network portion of point cloud learning model 700 (FIG. 7) and may exclude MLP 714 and segmentation network 702 of the point cloud learning model 700. For instance, point cloud learning model encoder 1002 may have the architecture of classification network 701 (e.g., as shown in FIG. 7, potentially omitting fully-connected network 714).

The input to decoder 1004 may include the values in global feature vector 713. In some examples, the input to decoder 1004 may also include the n 64-dimensional points of array 709. Thus, in such examples, the input to decoder 1004 may be the same as input 706 of segmentation network 702 as shown in the example of FIG. 7. Based on this input, decoder 1004 may generate one or more output point clouds. In some examples, an output point cloud includes points corresponding to bone and points corresponding to an orthopedic prosthesis. In other words, the points in the output point cloud may include points representing a predicted prosthesis shape (e.g., all or a portion of the predicted prosthesis shape). In some examples, an output point cloud may include only points corresponding to the orthopedic prosthesis.

Decoder 1004 may be implemented in one of a variety of ways. For instance, decoder 1004 may include a series of levels, similar to levels 606 of FIG. 6. In some examples, the decoder branch may be implemented using one or more levels of MLPs. In some examples, decoder 1004 may sample N points in a unit square in 2-dimensions. Thus, decoder 1004 may randomly determine N points having x-coordinates in a range of [0,1] and y-coordinates in the range of [0,1]. Additionally, decoder 1004 may obtain a feature vector, such as global feature vector 713. Decoder 1004 may concatenate the feature vector and the sampled points to obtain a combined vector. Decoder 1004 may apply K MLPs (where K is an integer greater than or equal to 1) to the combined vector to generate a single point in an output point cloud. Each of the K MLPs may generate points in a different patch (e.g., area) of the output point cloud. Each of the MLPs may reduce the number of features from 1026 to 3. The 3 features may correspond to the 3 coordinates of a point of the output point cloud. For instance, for each sampled point n in N, the MLPs may reduce the features from 1026 to 512 to 256 to 128 to 64 to 3. Thus, a K×N×3 vector may contain the output point cloud. In some examples, as part of training the MLPs of decoder 1004, decoder 1004 may calculate a chamfer loss of an output point cloud relative to a ground-truth point cloud. Decoder 1004 may use the chamfer loss in a backpropagation process to adjust parameters of the MLPs.

In some examples, training unit 204 may use a point cloud learning model, such as point cloud learning model 700, that includes both a classification network (e.g., classification network 701) and a segmentation network (e.g., segmentation network 702) as part of a process to generate training datasets for training machine learning model 1000 of FIG. 10. For ease of explanation, this disclosure may refer to the point cloud learning model used for generating training datasets for training machine learning model 1000 as a “segmentation point cloud learning model.” For example, training unit 204 may generate an input point cloud for the segmentation point cloud learning model based on one or more post-implantation medical images, such as post-implantation medical images in surgical plans 120. Thus, in this example, the input point cloud for the segmentation point cloud learning model may include points representing one or more bones of a patient and one or more orthopedic prostheses implanted in the patient.

The post-implantation medical images may be selected from among the case files of well-trained and experienced surgeons. Therefore, the post-implantation medical images (and the resulting point clouds) may represent what a well-trained and experienced surgeon actually did during surgery. Training unit 204 may apply the segmentation point cloud learning model to segment the points of the input point cloud between points corresponding to bone and points corresponding to an implanted orthopedic prosthesis. Thus, the segmentation point cloud learning model may generate a segmented point cloud similar to FIG. 9. Training unit 204 may use the segmented point cloud as expected output data of a training dataset. Furthermore, training unit 204 may remove the points of the segmented point cloud that correspond to the implanted orthopedic prostheses. Training unit 204 may then use the resulting bone-only point cloud as the input data of the training dataset.

In other examples, training unit 204 may have access to pre- and post-surgical medical images in surgical plans 120. As in the example above, these pre- and post-surgical medical images may be selected from among the case files of well-trained and experienced surgeons. Training unit 204 may generate point clouds based on the pre-surgical medical images and point clouds based on the post-surgical medical images. In such examples, training unit 204 may use a point cloud based on a pre-surgical medical image as the input data of the training dataset. In some examples, the pre-surgical medical image is a CT-based image, such as a CT volume scan of a knee, ankle, or other portion of the patient's anatomy. Training unit 204 may then use a segmentation point cloud learning model to classify points in the post-surgical point cloud as corresponding to bone or corresponding to an orthopedic prosthesis. Training unit 204 may use the segmented point cloud generated by the segmentation point cloud learning model as the expected output data of the training dataset. In some examples, training unit 204 may modify the segmented point cloud to remove points corresponding to bone, while leaving points corresponding to the orthopedic prosthesis in the segmented point cloud for use as the expected output data of the training dataset.

In this way, for at least one specific training dataset of a plurality of training datasets, training unit 204 may generate a pre-surgical point cloud based on one or more pre-surgical images of a historic patient in a plurality of historic patients. Training unit 204 may also generate a post-surgical point cloud based on one or more post-surgical medical images of the historic patient. Training unit 204 may apply a point cloud learning model to the post-surgical point cloud to generate a segmented point cloud in which points of the post-surgical point cloud are classified as corresponding to bone or corresponding to an orthopedic prosthesis of the historic patient. In this example, the pre-surgical point cloud is the input point cloud of the specific training dataset and the expected output point cloud of the specific training dataset includes at least the points of the segmented point cloud corresponding to the orthopedic prosthesis of the historic patient.

After generating a plurality of training datasets, training unit 204 may use the training datasets to perform a training process. The training process may include applying machine learning model 1000 using the input data of a training dataset, comparing the resulting output of machine learning model 1000 to the expected output data of the training dataset using a cost function, and performing a backpropagation operation to adjust parameters (e.g., weights) of machine learning model 1000 based on the comparison.

In this way, training unit 204 may generate a plurality of training datasets. For each respective training dataset of the plurality of training datasets, the respective training dataset may correspond to a respective historic patient of a plurality of historic patients, the respective training dataset may include an input point cloud that includes points corresponding to the bone of the respective historic patient, and the respective training dataset includes an expected output point cloud that includes points corresponding to a planned orthopedic prosthesis that was planned for attachment to the bone of the respective historic patient. Training unit 204 may train the machine learning model based on the training datasets.

The segmentation point cloud learning model may itself need to be trained using point clouds that are manually segmented.

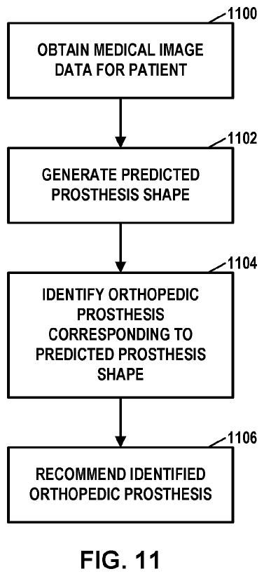

FIG. 11 is a flowchart illustrating an example operation in accordance with one or more techniques of this disclosure. In the example of FIG. 9, planning system 118 may obtain medical image data for the patient (1100). For example, planning system 118 may obtain a plurality of CT images, MRI images, or other types of images. For instance, planning system 118 may receive the medical image data (e.g., CT images, MRI images, etc.) from imaging system 116). In other instances, planning system 118 may receive the medical image data, e.g., from surgical plans 120. In some examples, planning system 118 may obtain a point cloud. In some such examples, planning system 118 may generate the point cloud based on a plurality of CT images, MRI images, or other types of images. In some examples, planning system 118 may receive the point cloud from another system.

Additionally, planning system 118 may apply a machine learning model 200 to generate a predicted prosthesis shape for the patient based on the medical image data for the patient (1102). In some examples where the medical image data for the patient includes a plurality of CT images, each of the CT images has a width dimension and a height dimension, and each of the CT images corresponds to a different depth-dimension layer in a plurality of depth-dimension layers. In such examples, as part of applying machine learning model 200 to generate the predicted prosthesis shape for the patient, prediction unit 202 of planning system 118 may provide the CT images as input to machine learning model 200. Prediction unit 202 may obtain a plurality of output images generated by machine learning model 200. Each respective output image of the plurality of output images corresponds to a respective depth-dimension layer in the plurality of depth-dimension layers and shows a profile of the predicted prosthesis shape in the respective depth-dimension layer.

In examples where planning system 118 obtains a point cloud that includes points corresponding to bones of the patient, prediction unit 202 may, as part of applying machine learning model 200 to generate the predicted prosthesis shape for the patient, provide the point cloud (i.e., a first point cloud) as input to machine learning model 200 (e.g., a point cloud learning model). Prediction unit 202 may obtain a second point cloud from machine learning model 200. The second point cloud model includes points arranged in the predicted prosthesis shape for the patient.

Furthermore, planning system 118 may identify, from a plurality of orthopedic prostheses for implantation in the patient (e.g., orthopedic prostheses in catalog 122), an orthopedic prosthesis that corresponds to the predicted prosthesis shape (1104). For example, recommendation unit 206 of planning system 118 may perform an ICP algorithm or Nelder-Mead algorithm to align the predicted prosthesis shape with the respective orthopedic prosthesis. Recommendation unit 206 may then determine a similarity metric for the respective orthopedic prosthesis that indicates a similarity between the aligned predicted prosthesis shape and the respective orthopedic prosthesis. Recommendation unit 206 may then identify the orthopedic prosthesis based on the similarity metrics for the orthopedic prostheses. In some examples, planning system 118 may use a Dice metric as the similarity metric. The Dice metric may range from 0 to 1, where 1 indicates a perfect match.

In some examples, planning system 118 may identify the orthopedic prosthesis based on a similarity metric and based on other clinical information. For instance, planning system 118 may determine fitness scores for a plurality of orthopedic prostheses based on similarity metrics and other clinical information. Planning system 118 may rank the orthopedic prostheses based on their fitness scores and identify the highest-ranking orthopedic prosthesis. The other clinical information may include information such as placement of the implant in a most suitable anatomical position. For instance, planning system 118 may determine the fitness score for a knee orthopedic prosthesis based on the similarity metric and a score characterizing a degree to which the knee orthopedic prosthesis minimizes notching in a femur.

In some examples, planning system 118 may determine a recommended position of the orthopedic prosthesis. For example, machine learning model 200 may generate a 3D image showing a predicted prosthesis shape. The predicted prosthesis shape is shown in the 3D image at a particular position (e.g., location and orientation). Planning system 118 perform a process to align a 3D image of the identified orthopedic prostheses with the predicted prosthesis shape. The resulting position of the 3D image of the identified orthopedic prosthesis may be the recommended position of the orthopedic prosthesis. In some examples, planning system 118 may obtain a 3D point cloud of the identified orthopedic prosthesis and a 3D point cloud of the predicted prosthesis shape. Planning system 118 may align the 3D point cloud of the identified orthopedic prosthesis with the 3D point cloud of the predicted prosthesis shape, e.g., using a Nelder-Mead method. The resulting position of the 3D point cloud of the identified prostheses may be the recommended position of the orthopedic prosthesis. The recommend position of the orthopedic prosthesis may be expressed in terms of a 3D rigid body transformation matrix.

In some examples, the plurality of orthopedic prostheses (e.g., the orthopedic prostheses in catalog 122) may be limited to a predefined type of orthopedic prosthesis. In some examples, the predefined type of orthopedic prosthesis may be selected by a surgeon. In some examples, planning system 118 may predict a size of the orthopedic implant for the patient separately from identifying the orthopedic implant or a recommended position of the orthopedic prosthesis. For instance, planning system 118 may apply a machine learning model (e.g., a neural network or other type of machine learning mode), a set of business rules, or another type of system to determine the size the orthopedic prosthesis. In an example regarding Total Ankle Replacement (TAR), a machine learning model for predicting a prosthesis size (e.g., a MLP size prediction model) may take as inputs both an antero-posterior length and a medio-lateral width of a tibia diaphysis at different heights (where the prosthesis is supposed to be implanted). This information describes the morphology of the patient's tibia, thereby enabling the prediction of the prosthesis size. The machine learning model may thus be trained to link an implant size to the patient tibia morphology. In this way, the orthopedic prosthesis may be fully defined in terms of type and size and prediction unit 202 may be able to register the orthopedic prosthesis as a point cloud to a 3D point cloud of the predicted prosthesis shape. The registration process may generate data representing transforms between a coordinate frame of orthopedic prosthesis and the 3D point cloud of the predicted prosthesis shape. In other examples, a patient-specific orthopedic prostheses may be manufactured based on the predicted prosthesis shape.

Planning system 118 may recommend the identified orthopedic prosthesis (1106). For example, planning system 118 may output a message that indicates the identified orthopedic prosthesis. For instance, the message may indicate a part number of name of the identified orthopedic prosthesis. Furthermore, in some examples, planning system 118 may indicate an orientation of the identified orthopedic prosthesis when implanted in the patient. Planning system 118 may determine the orientation of the identified orthopedic prosthesis so that the identified orthopedic prosthesis has the same orientation as the predicted prosthesis shape, e.g., in a generated image or point cloud. In some examples, planning system 118 may provide instructions or online features to a user on how to order the identified orthopedic prosthesis.

FIG. 12 is a conceptual diagram illustrating an example U-Net CNN 1200 in accordance with one or more techniques of this disclosure. In the example of FIG. 12, medical image data may include a 3D image of one or more bones of the patient, such as the bones of the ankle, knee, shoulder, or other parts of the anatomy of the patient. Prediction unit 202 (or another system) may generate the 3D image based on a stack of 2D images (e.g., CT images) arranged along a depth dimension. The location of each pixel in the stack of 2D images may be a voxel. Thus, the 3D image may be a CT volume scan. In the example of FIG. 12, each of the cuboids may represent a multi-dimensional array. For each of the cuboid, the number above the cuboid indicates an example number of dimensions of the multi-dimensional array represented by the cuboid.

In the example of FIG. 12, a left branch of the “U” shape of U-Net CNN may be referred to as an “encoder.” The encoder extracts high level features in a 3D input image. A right branch of the “U” shape may be referred to as a “decoder.” The decoder reconstructs information at a voxel level. An output of U-Net CNN 1200 may include a 3D output image that includes a 3D predicted prosthesis shape. The 3D output image may have the same size as the 3D input image. In other words, for each voxel of the input image, U-Net CNN 1200 may output a corresponding voxel.

In the example of FIG. 12, the encoder is composed of a succession of convolution, batch normalization, and activation (“ReLU”) layers (represented in FIG. 12 as white filled triangles), and down sampling operations (represented in FIG. 12 as down-forward pointing arrows) at each of levels 1202A-1202C (collectively, “levels 1202”). A transition level 1204 between the levels 1202 of the encoder includes a series of convolution layers. The convolutions at each level may use a 3×3×3 filter. In other examples, the number of channels, number of levels, filter size, and stride length may be different. For instance, in one example, the U-Net CNN 1200 may include 5 layers with 2 residual units of 2 convolutions each. A “residual unit” is a successive application of convolution, normalization, and parametric rectified linear unit for activation function. The last layer of the residual unit sums the input to the residual unit with the output of the residual unit. In this example, each of the 5 layers may have skip connections (e.g., bridges) between them.

The decoder may be symmetrically built where the down sampling operations are replaced by “Up” convolutions, which may include doubling the size of the feature maps at each level of levels 1206A-1206C (collectively, “levels 1206”). In the example of FIG. 12, U-net CNN 1200 includes bridges 1208 that enable connections between some high-level features that are extracted to help the reconstruction at the voxel level in the decoder. Thus, the input arrays for each of levels 1206 may be a concatenation of data generated by an up-convolution layer and data derived from a corresponding level of the encoder. As shown in the example of FIG. 12, a final layer of level 1206A of the decoder may be a convolution layer (represented in FIG. 12 as a black-filled triangle). The output of the decoder may include a plurality of output images corresponding to a respective depth-dimension layer in the plurality of depth-dimension layers and may show a profile of the predicted prosthesis shape in the respective depth-dimension layer. In some examples, the output of the decoder is binary mask in which voxels are labeled as being part of prosthesis shape or part of the background.

In some examples, the output of the decoder includes numerical score values for voxels. The score value for a voxel may indicate an amount of space within the voxel that corresponds to the orthopedic prosthesis. For instance, the score value for a voxel may be 100 if an orthopedic prosthesis defined by the predicted prosthesis shape occupies 100% of the voxel. The score value for a voxel may be 50 if the orthopedic prosthesis defined by the predicted prosthesis shape occupies 50% of the voxel, and so on. Including such numerical score values for voxels may provide a more precise anatomical volume for the orthopedic prosthesis.

In some examples, additional data may be included in the input to level 1206C of U-Net CNN 1200. For instance, information regarding biomechanics of the patient may be included in the input to level 1206C. Including the information regarding the biomechanics may help U-Net CNN 1200 generate predicted prosthesis shapes appropriate for the biomechanics of individual patients.

Training unit 204 may train U-Net CNN 1200 or similar machine learning models. For example, training unit 204 may generate a plurality of training datasets. For each respective training dataset of the plurality of training datasets, the respective training dataset may correspond to a respective historic patient of a plurality of historic patients. Furthermore, the respective training dataset may include a respective set of images showing the bone of the respective historic patient. The respective training dataset includes a set of expected images showing a planned orthopedic prosthesis that was planned for attachment to the bone of the respective historic patient. In some examples, the expected images are post-implantation prosthesis binary masks that each represent a shape (and, in some examples, position) of a planned or implanted orthopedic prosthesis.

Training unit 204 may train machine learning model 200 (e.g., CNN 600, U-Net CNN 1200) based on the training datasets. In some examples, the training datasets may be divided into training sets, validation, sets, and test sets (e.g., according to an 8:1:1 ratio) and shuffled. Training unit 204 may evaluate a production version of machine learning model 200 using the test set to produce an estimate of performance of machine learning model 200.

While the techniques been disclosed with respect to a limited number of examples, those skilled in the art, having the benefit of this disclosure, will appreciate numerous modifications and variations there from. For instance, it is contemplated that any reasonable combination of the described examples may be performed. It is intended that the appended claims cover such modifications and variations as fall within the true spirit and scope of the invention.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Operations described in this disclosure may be performed by one or more processors, which may be implemented as fixed-function processing circuits, programmable circuits, or combinations thereof, such as one or more digital signal processors (DSPs), general purpose microprocessors, application specific integrated circuits (ASICs), field programmable gate arrays (FPGAs), or other equivalent integrated or discrete logic circuitry. Fixed-function circuits refer to circuits that provide particular functionality and are preset on the operations that can be performed. Programmable circuits refer to circuits that can programmed to perform various tasks and provide flexible functionality in the operations that can be performed. For instance, programmable circuits may execute instructions specified by software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. Accordingly, the terms “processor” and “processing circuitry,” as used herein may refer to any of the foregoing structures or any other structure suitable for implementation of the techniques described herein.

Various examples have been described. These and other examples are within the scope of the following claims.

1. A method for automatically recommending an orthopedic prosthesis for implantation in a patient, the method comprising:
obtaining, by a computing system, medical image data for the patient;
applying, by the computing system, a machine learning model to generate a predicted prosthesis shape for the patient based on the medical image data for the patient;
identifying, by the computing system, from data regarding a plurality of orthopedic prostheses available for implantation in the patient, an orthopedic prosthesis that corresponds to the predicted prosthesis shape; and
recommending, by the computing system, the identified orthopedic prosthesis for implantation in the patient.

2. The method of claim 1, wherein:
the medical image data for the patient comprises a plurality of input images, each of the input images having a width dimension and a height dimension, and each of the input images corresponding to a different depth-dimension layer in a plurality of depth-dimension layers, and
applying the machine learning model to generate the predicted prosthesis shape for the patient comprises:
providing, by the computing system, the input images as input to the machine learning model; and
obtaining, by the computing system, a plurality of output images generated by the machine learning model, each respective output image of the plurality of output images corresponding to a respective depth-dimension layer in the plurality of depth-dimension layers and showing a profile of the predicted prosthesis shape in the respective depth-dimension layer.

3. The method of claim 2, further comprising:
generating, by the computing system, a plurality of training datasets, wherein for each respective training dataset of the plurality of training datasets:
the respective training dataset corresponds to a respective historic patient of a plurality of historic patients,
the respective training dataset includes a respective set of input images showing a bone of the respective historic patient, and
the respective training dataset includes a set of expected images showing a planned orthopedic prosthesis that was planned for attachment to the bone of the respective historic patient; and
training, by the computing system, the machine learning model based on the training datasets.

4. The method of claim 3, wherein the plurality of training datasets corresponds to a knee alignment technique.

5. The method of claim 1, wherein identifying the orthopedic prosthesis comprises:
for each respective orthopedic prosthesis of the plurality of orthopedic prostheses:
performing, by the computing system, a registration process that aligns the predicted prosthesis shape with the respective orthopedic prosthesis; and
determining, by the computing system, a similarity metric for the respective orthopedic prosthesis that indicates a similarity between the aligned predicted prosthesis shape and the respective orthopedic prosthesis; and
identifying, by the computing system, the identified orthopedic prosthesis based on the similarity metrics for the orthopedic prostheses.

6. The method of claim 1, wherein the machine learning model has a U-Net or V-Net architecture.

7. The method of claim 1, wherein the machine learning model has a U-net architecture having 5 layers with 2 residual units of 2 convolutions each.

8. The method of claim 1, wherein the orthopedic prosthesis is for implantation in a bone of a knee of the patient.

9. The method of claim 1, wherein:
obtaining the medical image data for the patient comprises obtaining, by the computing system, a first point cloud that includes points corresponding to bones of the patient; and
applying the machine learning model to generate the predicted prosthesis shape for the patient comprises:
providing, by the computing system, the first point cloud as input to the machine learning model; and
obtaining, by the computing system, a second point cloud from the machine learning model, wherein the second point cloud includes points arranged in the predicted prosthesis shape for the patient.

10. The method of claim 9, further comprising:
generating, by the computing system, a plurality of training datasets, wherein for each respective training dataset of the plurality of training datasets:
the respective training dataset corresponds to a respective historic patient of a plurality of historic patients,
the respective training dataset includes an input point cloud that includes points corresponding to a bone of the respective historic patient, and
the respective training dataset includes an expected output point cloud that includes points corresponding to a planned orthopedic prosthesis that was planned for attachment to the bone of the respective historic patient; and
training, by the computing system, the machine learning model based on the training datasets.

11. The method of claim 10, wherein generating the plurality of training datasets comprises, for at least one specific training dataset of the plurality of training datasets:
generating a pre-surgical point cloud based on one or more pre-surgical images of a historic patient in the plurality of historic patients;
generating a post-surgical point cloud based on one or more post-surgical medical images of the historic patient; and
applying a point cloud learning model to the post-surgical point cloud to generate a segmented point cloud in which points of the post-surgical point cloud are classified as corresponding to bone or corresponding to an orthopedic prosthesis of the historic patient,
wherein the pre-surgical point cloud is the input point cloud of the specific training dataset and the expected output point cloud of the specific training dataset includes at least the points of the segmented point cloud corresponding to the orthopedic prosthesis of the historic patient.

12. The method of claim 1, wherein the machine learning model is trained based on medical images of historic surgeries that implanted orthopedic prostheses into historic patients.

13. The method of claim 1, wherein the method further comprises determining a recommended position of the orthopedic prosthesis.

14. The method of claim 1, wherein the machine learning model is a first machine learning model, the predicted prosthesis shape is a first predicted prosthesis shape, the first predicted prosthesis shape is a predicted shape of an orthopedic prosthesis in a first type of orthopedic protheses, the plurality of orthopedic prostheses is a first plurality of orthopedic prostheses, each orthopedic prosthesis in the first plurality of orthopedic prostheses is in the first type of orthopedic protheses, the identified orthopedic prosthesis is a first identified orthopedic prosthesis, and the method further comprises:
applying, by the computing system, a second machine learning model to generate a second predicted prosthesis shape for the patient based on the medical image data for the patient, wherein the second predicted prosthesis shape is a predicted shape of an orthopedic prosthesis in a second type of orthopedic prostheses;
identifying, by the computing system, from data regarding a second plurality of orthopedic prostheses available for implantation in the patient, a second orthopedic prosthesis that corresponds to the predicted prosthesis shape; and
recommending, by the computing system, the second identified orthopedic prosthesis for implantation in the patient.

15. The method of claim 4, wherein the second machine learning model was trained in part using transfer learning from the first machine learning model.

16. A computing system for automatically recommending an orthopedic prosthesis for implantation in a patient, the computing system comprising:
a storage system configured to store medical image data for the patient; and
processing circuitry configured to:
apply a machine learning model to generate a predicted prosthesis shape for the patient based on the medical image data for the patient;
identify, from a plurality of orthopedic prostheses available for implantation in the patient, an orthopedic prosthesis that corresponds to the predicted prosthesis shape; and
recommend the identified orthopedic prosthesis for implantation in the patient.

17. The computing system of claim 16, wherein:
the medical image data for the patient comprises a plurality of input images, each of the input images having a width dimension and a height dimension, and each of the input images corresponding to a different depth-dimension layer in a plurality of depth-dimension layers, and
the processing circuitry is configured to, as part of applying the machine learning model to generate the predicted prosthesis shape for the patient:
provide the input images as input to the machine learning model; and
obtain a plurality of output images generated by the machine learning model, each respective output image of the plurality of output images corresponding to a respective depth-dimension layer in the plurality of depth-dimension layers and showing a profile of the predicted prosthesis shape in the respective depth-dimension layer.

18. The computing system of claim 17, wherein the processing circuitry is further configured to:
generate a plurality of training datasets, wherein for each respective training dataset of the plurality of training datasets:
the respective training dataset corresponds to a respective historic patient of a plurality of historic patients,
the respective training dataset includes a respective set of input images showing a bone of the respective historic patient, and
the respective training dataset includes a set of expected images showing a planned orthopedic prosthesis that was planned for attachment to the bone of the respective historic patient; and
train the machine learning model based on the training datasets.

19. (canceled)

20. The computing system of claim 16, wherein the processing circuitry is configured to, as part of identifying the identified orthopedic prosthesis:
for each respective orthopedic prosthesis in the plurality of orthopedic prostheses:
perform a registration process that aligns the predicted prosthesis shape with the respective orthopedic prosthesis, and
determine a similarity metric for the respective orthopedic prosthesis that indicates a similarity between the aligned predicted prosthesis shape and the respective orthopedic prosthesis; and
identify the orthopedic prosthesis based on the similarity metrics for the orthopedic prostheses.

21. The computing system of claim 16, wherein the machine learning model has a U-Net or V-Net architecture.

22-23. (canceled)

24. The computing system of claim 16, wherein:
the processing circuitry is configured to, as part of obtaining the medical image data for the patient, obtain a first point cloud that includes points corresponding to bones of the patient;
the processing circuitry is configured to, as part of applying the machine learning model to generate the predicted prosthesis shape for the patient:
provide the first point cloud as input to the machine learning model; and
obtain a second point cloud from the machine learning model, wherein the second point cloud includes points arranged in the predicted prosthesis shape for the patient.

25. The computing system of claim 24, wherein the processing circuitry is further configured to:
generate a plurality of training datasets, wherein for each respective training dataset of the plurality of training datasets:
the respective training dataset corresponds to a respective historic patient of a plurality of historic patients,
the respective training dataset includes an input point cloud that includes points corresponding to a bone of the respective historic patient, and
the respective training dataset includes an expected output point cloud that includes points corresponding to a planned orthopedic prosthesis that was planned for attachment to the bone of the respective historic patient; and
train the machine learning model based on the training datasets.

26. The computing system of claim 25, wherein the processing circuitry is configured to, as part of generating the plurality of training datasets, for at least one specific training dataset of the plurality of training datasets:
generate a pre-surgical point cloud based on one or more pre-surgical images of a historic patient in the plurality of historic patients;
generate a post-surgical point cloud based on one or more post-surgical medical images of the historic patient; and
apply a point cloud learning model to the post-surgical point cloud to generate a segmented point cloud in which points of the post-surgical point cloud are classified as corresponding to bone or corresponding to an orthopedic prosthesis of the historic patient,
wherein the pre-surgical point cloud is the input point cloud of the specific training dataset and the expected output point cloud of the specific training dataset includes at least the points of the segmented point cloud corresponding to the orthopedic prosthesis of the historic patient.

27-31. (canceled)

32. A non-transitory computer-readable data storage medium comprising instructions that, when executed, cause a computing system to:
obtain medical image data for a patient;
apply a machine learning model to generate a predicted prosthesis shape for the patient based on the medical image data for the patient;
identify, from data regarding a plurality of orthopedic prostheses available for implantation in the patient, an orthopedic prosthesis that corresponds to the predicted prosthesis shape; and
recommend the identified orthopedic prosthesis for implantation in the patient.
